(19) United States
(12) Patent Application Publication
(10) Pub. No.: US 2026/0142005 A1
(43) Pub. Date: May 21, 2026

(54) MOTIVATIONAL ENGINE DEPLOYED ON MOBILE AND WEARABLE DEVICES

(71) Applicant: CARROT FERTILITY, INC., Menlo Park, CA (US)

(72) Inventors: Tammy Sun, Menlo Park, CA (US); Mikayla Johnson, Menlo Park, CA (US); Phil Libin, Bentonville, AR (US); Andrew Sinkov, Brooklyn, NY (US); Chris Ploeg, Oak Park, IL (US); Josh Parenti, San Francisco, CA (US); Kurt Libby, Bentonville, AR (US); Alex Pashintsev, Cupertino, CA (US); Natalia Galaktionova, Tbilisi, GA (US); Christina King, Madison, WI (US)

(21) Appl. No.: 19/241,853

(22) Filed: Jun. 18, 2025

Related U.S. Application Data
(60) Provisional application No. 63/722,974, filed on Nov. 20, 2024.

(51) Int. Cl. G16H 20/00; G16H 10/60; G16H 40/67

(52) U.S. Cl. CPC G16H 20/00; G16H 10/60; G16H 40/67

(57) ABSTRACT

Systems and methods for generating personalized motivational content based on user-specific data and goals are provided. Example techniques may include obtaining motivational data indicating a health goal for a user; based on the motivational data: associating the user with a motivational profile; and determining one or more recommended behaviors to achieve the indicated health goal; inputting the motivational profile and the one or more recommended behaviors into a motivation generation machine learning model to obtain motivational content; and presenting the motivational content to the user.

### FIELD OF THE INVENTION

The present disclosure generally relates to technologies associated with goal-driven personal health management systems, and more particularly, to applying machine learning techniques to dynamically generate personalized motivational content based on user-specific data and goals.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The need for personalized wellness solutions has substantially increased as more consumers focus on health and wellness. This growth in the wellness market is supported by technological and scientific advancements, leading to the development of wellness products like wearable devices and applications that track fitness and monitor sleep.

However, real-life implementation and application of existing behavior formation techniques often falls short when it comes to goal-oriented wellness management systems. Despite the interest in using user-specific data and Generative AI for customized wellness recommendations, the existing integration of personalized approaches into health management systems is complicated and lacks an effective motivational framework because it requires significant effort and attention from the users to interact with the system or to dynamically update the recommendations as the users'needs change. This has led to a demand for more intuitive and user-friendly approaches to health management that can more seamlessly integrate into the users' lifestyles.

### SUMMARY

In one aspect, a computer-implemented method for providing recommended actions to motivate a user is provided. The method may include (1) obtaining, by one or more processors, motivational data indicating a health goal for the user; (2) based on the motivational data, associating, by the one or more processors, the user with a motivational profile and determining, by the one or more processors, one or more recommended behaviors to achieve the indicated health goal; (3) inputting, by the one or more processors, the motivational profile and the one or more recommended behaviors into a motivation generation machine learning model to obtain motivational content; and (4) presenting, via the one or more processors, the motivational content to the user. The method may include additional, less, or alternate actions, including those discussed elsewhere herein.

In another aspect, a computer system for generating personalized motivational content based on user-specific data and goals is provided. The computer system may include one or more processors and a memory storing computer-executable instructions that, when executed by the one or more processors, cause the one or more processors to: (1) obtain motivational data indicating a health goal for the user; (2) based on the motivational data, associate the user with a motivational profile and determine one or more recommended behaviors to achieve the indicated health goal; (3) input the motivational profile and the one or more recommended behaviors into a motivation generation machine learning model to obtain motivational content; and (4) present the motivational content to the user. The system may include additional, less, or alternate functionality, including that discussed elsewhere herein.

In still another aspect, a non-transitory computer-readable storage medium storing computer-readable instructions for generating personalized motivational content based on user-specific data and goals is provided. The computer-readable instructions, when executed by one or more processors, cause the one or more processors to: (1) obtain motivational data indicating a health goal for the user; (2) based on the motivational data, associate the user with a motivational profile and determine one or more recommended behaviors to achieve the indicated health goal; (3) input the motivational profile and the one or more recommended behaviors into a motivation generation machine learning model to obtain motivational content; and (4) present the motivational content to the user. The instructions may direct additional, less, or alternative functionality, including that discussed elsewhere herein.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 depicts an example computer system in which methods and systems for generating personalized motivational content based on user-specific data and goals are implemented, according to one embodiment.

FIG. 2 depicts a combined block and logic diagram in which example computer-implemented methods and systems for generating personalized motivational content based on user-specific data and goals are implemented, according to one embodiment.

FIG. 3 depicts a combined block and logic diagram in which example computer-implemented methods and systems for obtaining motivational data are implemented, according to one embodiment.

FIG. 4 depicts a combined block and logic diagram in which example computer-implemented methods and systems for associating a user with a motivational profile based on the motivational data are implemented, according to one embodiment.

FIG. 5 depicts a combined block and logic diagram in which example computer-implemented methods and systems for determining recommended behaviors based on the motivational profile are implemented, according to one embodiment.

FIG. 6 depicts a combined block and logic diagram in which example computer-implemented methods and systems for generating user-specific motivational content based on the recommended behaviors are implemented, including an example motivational content prompt, according to one embodiment.

FIG. 7 depicts a combined block and logic diagram in which example computer-implemented methods and systems for updating the motivational content based on compliance data are implemented, according to one embodiment.

FIG. 8 depicts an example user device display of the motivational content, according to one embodiment.

FIG. 9 depicts a flow diagram of an example computer-implemented method for generating personalized motivational content based on user-specific data and goals, according to one embodiment.

FIG. 10 depicts an example flow diagram for how the techniques described herein may be implemented, according to one embodiment.

FIG. 11 depicts an example flow diagram for how the techniques described herein may be implemented on a daily schedule, according to one embodiment

While the systems and methods disclosed herein is susceptible of being embodied in many different forms, it is shown in the drawings and will be described herein in detail specific example embodiments thereof, with the understanding that the present disclosure is to be considered as an exemplification of the principles of the systems and methods disclosed herein and is not intended to limit the systems and methods disclosed herein to the specific embodiments illustrated. In this respect, before explaining at least one embodiment consistent with the present systems and methods disclosed herein in detail, it is to be understood that the systems and methods disclosed herein is not limited in its application to the details of construction and to the arrangements of components set forth above and below, illustrated in the drawings, or as described in the examples.

Methods and apparatuses consistent with the systems and methods disclosed herein are capable of other embodiments and of being practiced and carried out in various ways. Also, it is to be understood that the phraseology and terminology employed herein, as well as the abstract included below, are for the purposes of description and should not be regarded as limiting.

### DETAILED DESCRIPTION

### Overview

The embodiments described herein relate to generating personalized motivational content with recommended actions tailored to motivate users to achieve their health goals. This approach leverages the power of machine learning and generative artificial intelligence (Gen AI) models to process motivational data, which includes but is not limited to user health data, content consumption and creation data, as well as data automatically captured by applications such as fitness and location tracking applications. By obtaining and analyzing the motivational data, the computer-implemented methods and systems associate users with motivational profiles and determine recommended behaviors aimed at achieving specified health goals based on their associated motivations. These profiles and behaviors are then input into a motivation generation machine learning model to produce motivational content that is tailored to the individual's motivations. This content is then presented to the user in a user-specific format, such as images, videos, websites, and/or audio recordings.

One challenge in the realm of personal health management has been the development of systems that not only track health metrics but also actively engage and motivate users to pursue healthier lifestyles. Conventional techniques often rely on static content that fails to consider the evolving nature of an individual's health goals, motivations, and circumstances. On the other hand, the instant techniques relate to dynamically updating motivational content in response to user feedback and progress, ensuring that the motivation remains relevant and effective over time. Additionally, the instant techniques relate to presenting the motivational content in a manner that aligns with the user's motivations to improve the likelihood the user performs the motivated activities. As a result, the user is provided with suggestions in a manner the involves less significant user effort and attention.

Additionally, techniques disclosed herein relate to a network of computing models (e.g., a profile generation model, a behavior generation model, and a motivation generation model) that configured to interact with one another in a particular manner that ensures that the motivational content presented to the user is relevant and likely to encourage the user to perform the motivated activity. More particularly, these models are interconnected in a manner that ensures that the right information is input into the right model at the right time to ensure that the motivational content is relevant and timely. By utilizing these models to analyze and process user data, the system can quickly and accurately identify the most effective motivational strategies for individual users. This not only streamlines the process of generating personalized motivational content but also ensures that the content is highly relevant and likely to engage the user effectively.

Furthermore, the techniques improve memory usage within computing systems. By employing machine learning models that adapt and learn from user feedback, the system can refine the motivational content and strategies over time without the need for storing large volumes of historical data. This dynamic learning approach allows for the efficient use of memory resources to retain the most relevant and current data for ongoing analysis and content generation.

### Example System

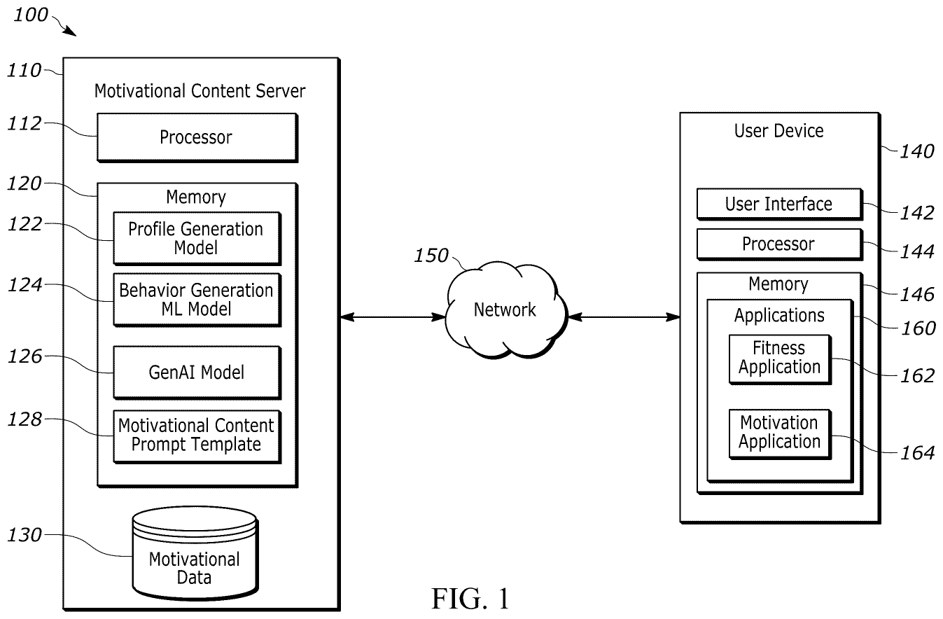

Referring now to the drawings, FIG. 1 depicts an example computer system 100 for generating personalized motivational content, according to one embodiment. The example system 100 may include both hardware and software components, as well as various data communications channels for communicating data between the various hardware and software components, as is described below.

The system 100 may include a motivational content server 110 as well as one or more user devices 140. Each of the user devices 140 may include, e.g., smart phones, smart watches or fitness tracker devices, tablets, laptops, virtual reality headsets, smart or augmented reality glasses, wearables, other personal computers, etc.

The user device(s) 140 may include, or may be configured with, a user interface 142 via which the user device 140 receives input from users and/or provides audible or visible output to users. For example, the user interface may include a display screen via which one or more graphical user interface (GUIs) are provided for presenting motivational content.

Additionally, the user device(s) 140 may include one or more processor(s) 144, as well as one or more computer memories 146. Memories 146 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 146 may store an operating system (OS) (e.g., iOS, Microsoft Windows, Linux, UNIX, etc.) capable of facilitating the functionalities, apps, methods, or other software as discussed herein. Memorie(s) 146 may also store a plurality of applications 160, including fitness application 162 (e.g., a fitness tracker that monitors fitness activity performed by a user) and motivation application 164 (e.g., an application configured to interface with the motivational content server 110 to, for example, configure a motivational profile of the user and obtain motivational content).

The user device 140 and the motivational content server 110 may be configured to communicate with one another via a wired or wireless computer network 150. Although one motivational content server 110, one user device 140, and one network 150 are shown in FIG. 1, any number of such motivational content servers 110, user devices 140, and networks 150 may be included in various embodiments.

The network 150 may comprise any suitable network or networks, including a local area network (LAN), wide area network (WAN), Internet, or combination thereof. For example, the network 150 may include a wireless cellular service (e.g., 4G, 5G, etc.). Generally, the network 150 enables bidirectional communication between the motivational content server 110 and one or more user devices 140. In one aspect, the network 150 may comprise a cellular base station, such as cell tower(s), communicating to the one or more components of the system 100 via wired/wireless communications based upon any one or more of various mobile phone standards, including NMT, GSM, CDMA, UMTS, LTE, 5G, or the like. Additionally or alternatively, the network 150 may comprise one or more routers, wireless switches, or other such wireless connection points communicating to the components of the system 100 via wireless communications based upon any one or more of various wireless standards, including by non-limiting example, IEEE 802.11a/b/c/g (Wi-Fi), Bluetooth, and/or the like. To facilitate such communications, the motivational content server 110 and user devices 140 may each include one or more wireless transceivers to receive and transmit wireless communications via the network 150.

In some embodiments, the motivational content server 110 is part of a cloud computing network or may otherwise communicate with other hardware or software components within one or more cloud computing environments to send, retrieve, or otherwise analyze data or information described herein. For example, in certain aspects of the present techniques, the system 100 may comprise an on-premise computing environment, a multi-cloud computing environment, a public cloud computing environment, a private cloud computing environment, and/or a hybrid cloud computing environment. For example, an entity (e.g., a business) may host one or more services in a public cloud computing environment (e.g., Alibaba Cloud, Amazon Web Services (AWS), Google Cloud, IBM Cloud, Microsoft Azure, etc.). The public cloud computing environment may be a traditional off-premise cloud (i.e., not physically hosted at a location owned/controlled by the business). Alternatively, or in addition, aspects of the public cloud may be hosted on-premise at a location owned/controlled by the business. The public cloud may be partitioned using visualization and multi-tenancy techniques and may include one or more infrastructure-as-a-service (IaaS) and/or platform-as-a-service (PaaS) services.

In some embodiments the motivational content server 110 may comprise one or more servers, which may comprise multiple, redundant, or replicated servers as part of a server farm. In still further aspects, such server(s) may be implemented as cloud-based servers, such as a cloud-based computing platform. For example, such server(s) may be any of the above-described services. The motivational content server 110 may include one or more processor(s) 112 (e.g., CPUs), one or more computer memories 120 as well as a motivational database 130. In one embodiment, as depicted in FIG. 1, the database 130 may be maintained at the motivational content server 110. Alternatively, the database 130 may be maintained externally and/or maintained across multiple database systems (including cloud storage systems). In these embodiments, the motivational content server 110 may be communicatively coupled to the database 130 via network 150.

The database 130 may be a relational database, such as Oracle, DB2, MySQL, a NoSQL based database, such as MongoDB, or another suitable database. The database 130 may store data that is used to train and/or operate one or more ML models, provide AR models/displays, among other things.

Memorie(s) 120 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 122 may store an operating system (OS) (e.g., Microsoft Windows, Linux, UNIX, etc.) capable of facilitating the functionalities, apps, methods, or other software as discussed herein.

Memorie(s) 120 may store a profile generation model 122, a behavior generation machine learning model 124, a Generative AI model 126, and/or a motivational content prompt template 128. Additionally, or alternatively, the memorie(s) 120 may store motivational data from various sources, such as from motivational database 130.

In various aspects, the models 122 124 may comprise a machine learning program or algorithm that may be trained by and/or employ a neural network, which may be a deep learning neural network, or a combined learning module or program that learns in one or more features or feature datasets in particular area(s) of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques.

Similarly, the Generative AI model 126 may be a machine learning program. For example, the

In some embodiments, the artificial intelligence and/or machine learning based algorithms used to train the models 122 and 124 may comprise a library or package executed on the motivational content server 110 (or other computing devices not shown in FIG. 1). For example, such libraries may include the TENSORFLOW based library, the PYTORCH library, and/or the SCIKIT-LEARN Python library.

Machine learning model(s) may be created and trained based upon example data (e.g., “training data”) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based upon the discovered rules, relationships, or model, an expected output.

According to aspects, the Generative AI model 126 may implement one or more machine learning models and/or training protocols therefor. For example, the Generative AI model 126 may implement one or more neural networks, deep learning models, decision trees, support vector machines, linear regression, generative AI models, reinforced learning models, random forests, Naïve Bayes models, large language models (LLMs), generative adversarial networks, foundation models, image recognition models, linear discriminant analysis models, creative applications, autoregressive models, supervised or unsupervised learning models, multimodal models, vision language models (VLMs), vision foundation models (VFMs), large multi-modal models (LMMs), Transformer models, or another machine learning or AI model for performing the methods described herein. It should be appreciated that the Generative AI model 126 may be configured to provide generated outputs in several different modalities (e.g., text, audio, visual, etc.) based on the user preferences.

Generally, the Generative AI model 126 may be configured to receive a prompt and provide generated content responsive to the prompt. For example, the memorie(s) 120 may include a motivation content generation prompt template 128 that forms the basis of the prompt input into the Generative AI model 126. The motivation content generation prompt template 128 may be a set of rules and/or instructions defining how the Generative AI model 126 is to generate motivational content. In some embodiments, the rules that form the motivation content generation prompt template 128 may be divided into subsets. For example, one set of rules may define a user motivations, another set of rules may define a recommended behavior to motivate, another set of rules may define how the analysis of the motivations and/or behaviors is to be performed, another set of rules may indicate compliance with prior motivational content, another set of rules may define how to present the motivational content.

It should be appreciated that the sets of rules related to user motivation and/or behaviors may be dynamically generated and/or selected based upon data associated with the user. Accordingly, the motivational content server 110 may populate the motivational content prompt template 128 with relevant sets of rules and/or instructions based upon an analysis of one or more data sources to generate the version of the prompt that is ultimately input into the Generative AI model 126.

In some embodiments, the models 122, 124 126 maintained at the motivational content server 110 may be front end applications to underlying machine learning models functionally maintained at other systems (e.g., a third party machine learning service provider). For example, the Generative AI model 126 may be configured to transmit prompts to the third-party system and receive outputs therefrom via network 150.

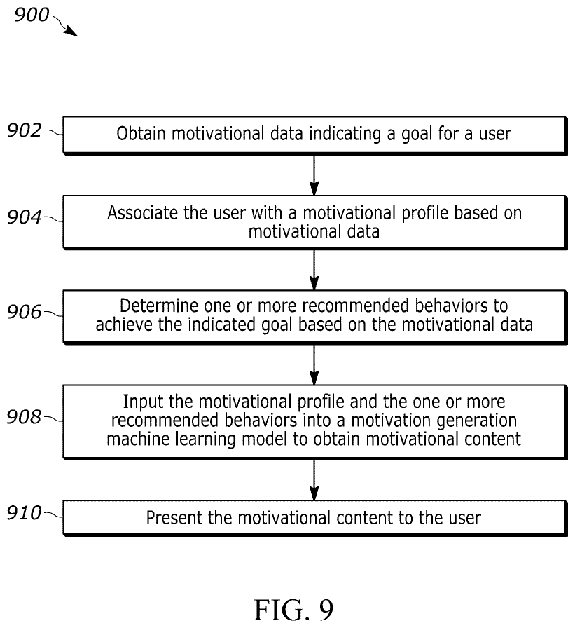

In addition, memorie(s) 120 may also store additional machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memorie(s) 120 may include instructions for carrying out any of the steps of the method 900 via an algorithm executing on the processors 112, which is described in greater detail below with respect to FIG. 9. It should be appreciated that one or more other applications may be envisioned and that are executed by the processor(s) 112. It should be appreciated that given the state of advancements of mobile computing devices, all of the processes functions and steps described herein may be present together on a mobile computing device.

### Example Workflow

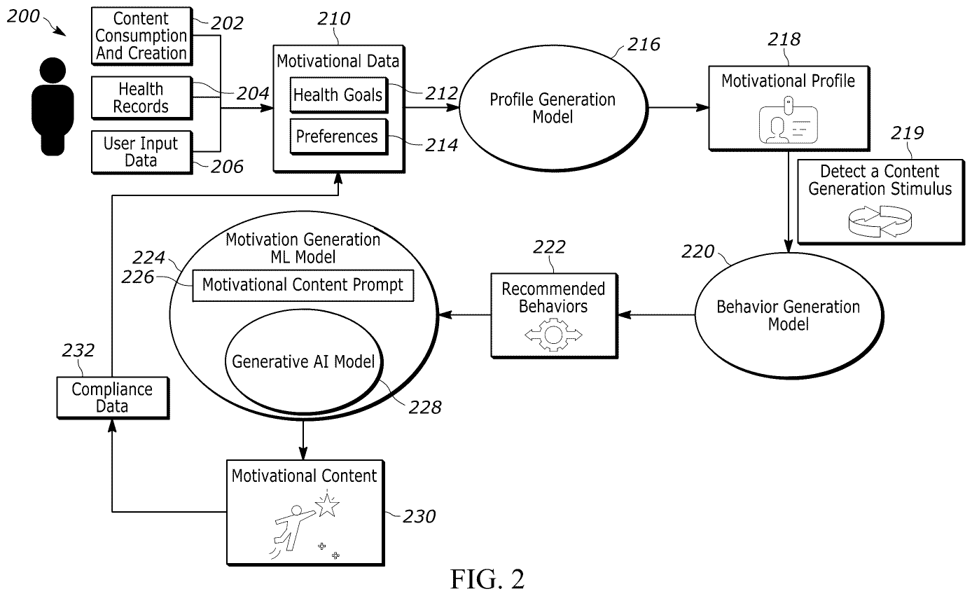

FIG. 2 depicts a combined block and logic diagram 200 for generating personalized motivational content based on user-specific data and goals, in which the techniques described herein may be implemented, according to some embodiments. The techniques described with respect to FIG. 2 may be implemented by a motivational content server (e.g., the motivational content server 110). Some of the blocks in FIG. 2 may represent hardware and/or software components of the motivational content server, other blocks may represent data structures or memory storing these data structures (e.g., 210), and other blocks may represent output data (e.g., 218).

As illustrated, in one embodiment, the motivational content server obtains, with the user's consent, motivational data 210 indicating a health goal 212 for a user. For example, a health goal 212 may be preparing for a significant sports event (e.g., running a marathon or participating in a multi-day bicycle race); planning a long-term travel to areas with different climatic and/or everyday life conditions; getting ready for fertility and maternity, having a healthier pregnancy and avoiding high-risk complications, with and without ART (Assistive Reproductive Technologies); improving postpartum recovery and outcomes, improving menopause-related symptoms and outcomes, recovering from a recent surgery; etc. In some embodiments, the health goals 212 may be predefined goals associated with respected sets of behaviors one would perform in furtherance of the goal. In these embodiments, the motivational content server may generate groupings of similar health goals 212 (e.g., fitness goals, fertility goals, wellness goals, etc.) that have similar behaviors. In other embodiments, the user may define a custom health goal 212. In these embodiments, the motivational content server may apply a neural network to identify one or more similar predefined health goals 212 that would include behaviors that may further the custom health goal. Alternatively, the motivational content server may consult a database of relevant documents (e.g., medical journals and/or published health data) and/or user-provided health records 204 to derive behaviors associated with the custom goal 212.

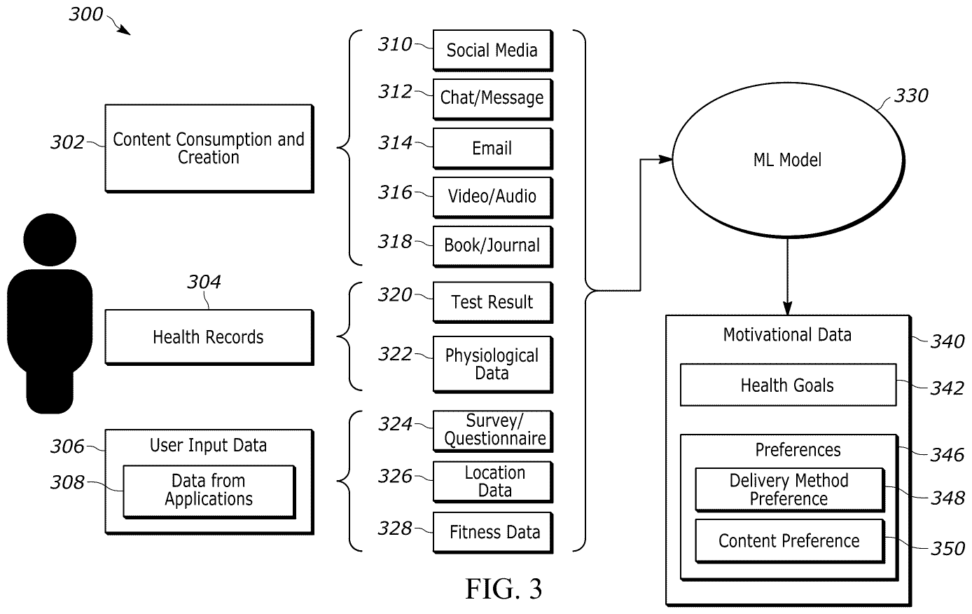

In one embodiment, the motivational content server may derive preferences 214 of the motivational data 210 from the user provided information. For example, the user's content consumption and creation 202 (e.g., social media content provided via a linked social media profile, media consumption data provided by a linked media consumption account (e.g., Netflix or Spotify)) may indicate topics that connect with the user. Accordingly, the motivational content server may analyze the user's content consumption and creation data 202 to identify such topics (e.g., particular TV shows, bands, books, brands, activities, etc.) that are important to the user such that the generated motivational content can be influenced from the topics to increase the likelihood the user performs the motivated behavior. In some embodiments, the user may provide the preference data 214 via the user's input data 206 (e.g., user data input via a GUI of a user device). More details about obtaining the motivational data 210 will be explained below with regards to FIG. 3.

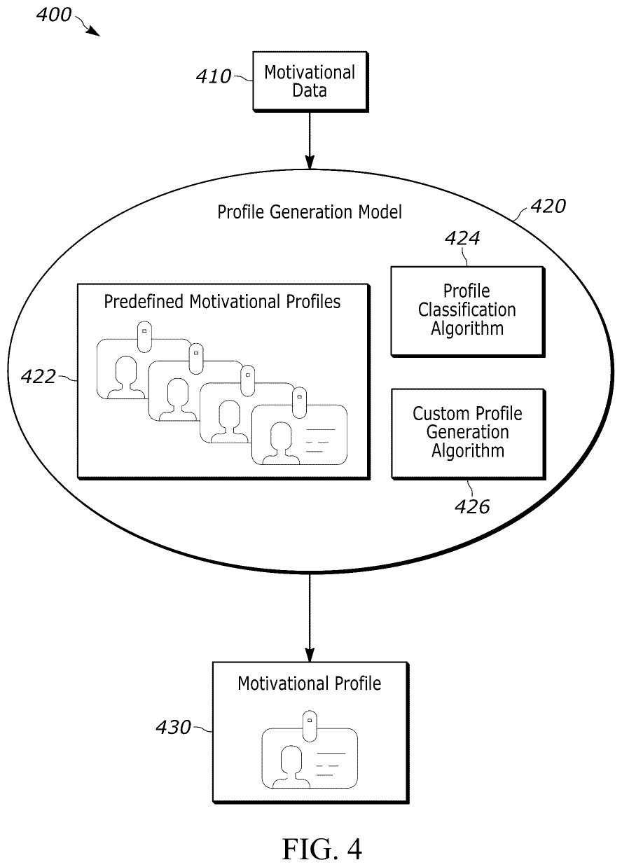

In the illustrated embodiment, the profile generation model 216 may be configured to analyze the motivational data 210 to associate the user with a motivational profile 218. In some embodiments, the motivational profiles 218 represent pre-defined personas and their respective personality traits. Additionally or alternatively, a customized motivational profile 218 may be generated based upon the motivational data 210. In some embodiments, customization may utilize a pre-defined persona as a baseline profile that is modified based on the motivational data 210. More details about the profile generation model process will be explained below with regards to FIG. 4.

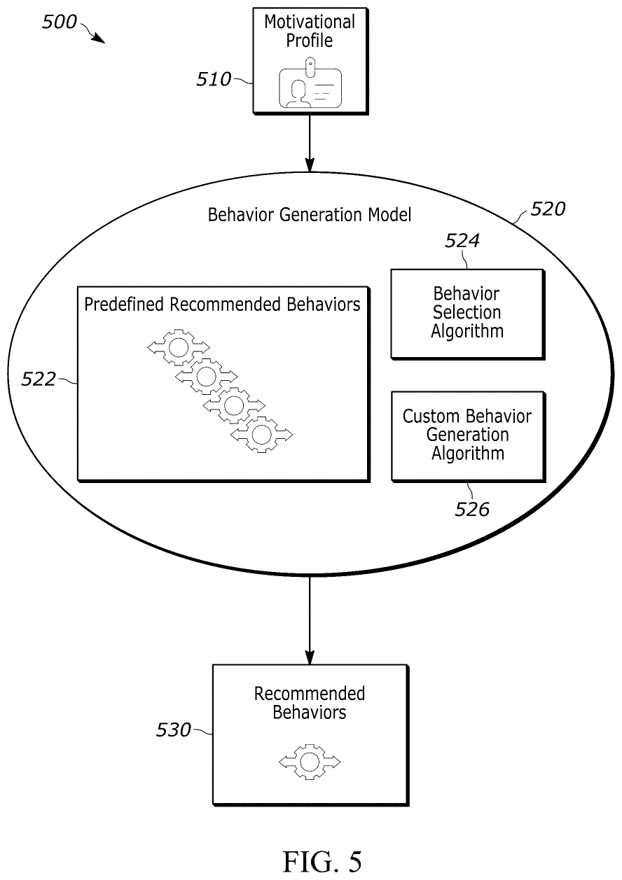

In response to a content generation stimulus 219, the motivational content server may input the motivational profile 218 into the behavior generation model 220 to determine one or more recommended behaviors 222. The content generation stimulus 219 may be a scheduled stimulus (e.g., every day at a particular time) or a dynamic stimulus (e.g., in response to a user interaction with a GUI). In some embodiments, the health goals 212 may be associated with sets of behaviors from which the behavior generation model can select the recommended behavior 222. More details about the behavior generation model process will be explained below with regards to FIG. 5.

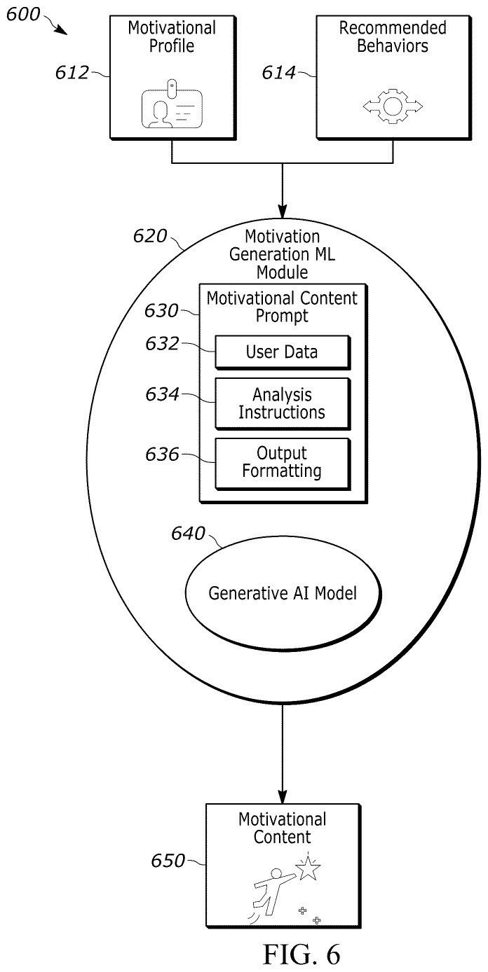

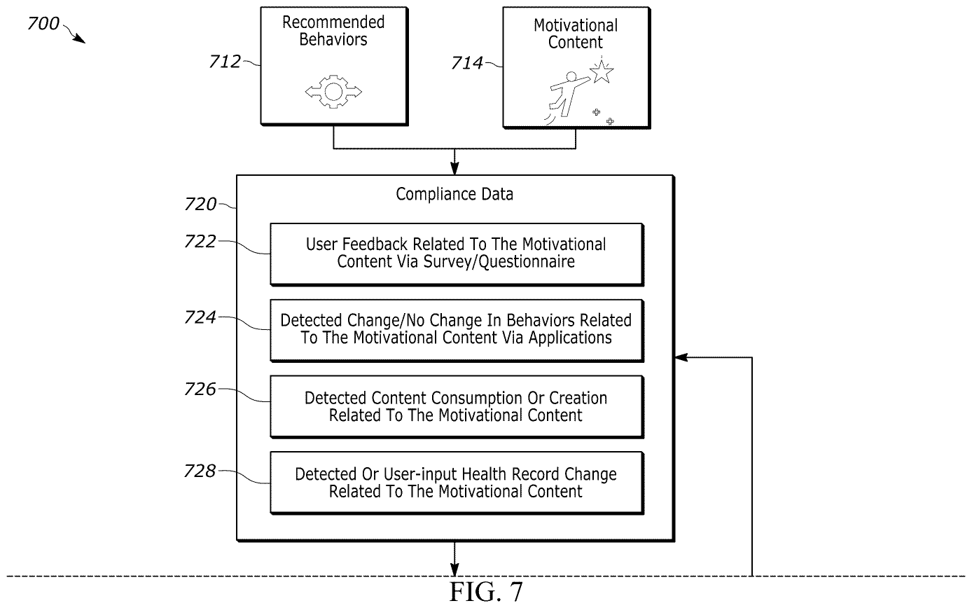

The motivational content server may then be configured to input the motivational profile 218 and the one or more recommended behaviors 222 into the motivation generation machine learning model 224 to generate motivational content 230 for presentation to the user. The motivation generation machine learning model 224 may be configured to generate a motivation content prompt 226 (e.g., by modifying a motivational content prompt template based on the motivational profile and the recommended behaviors 222). The motivation generation machine learning model may then input the motivation content prompt into a Generative AI model 228 to obtain the motivational content 230. More details about the motivation generation machine learning model process will be explained below with regards to FIG. 6.

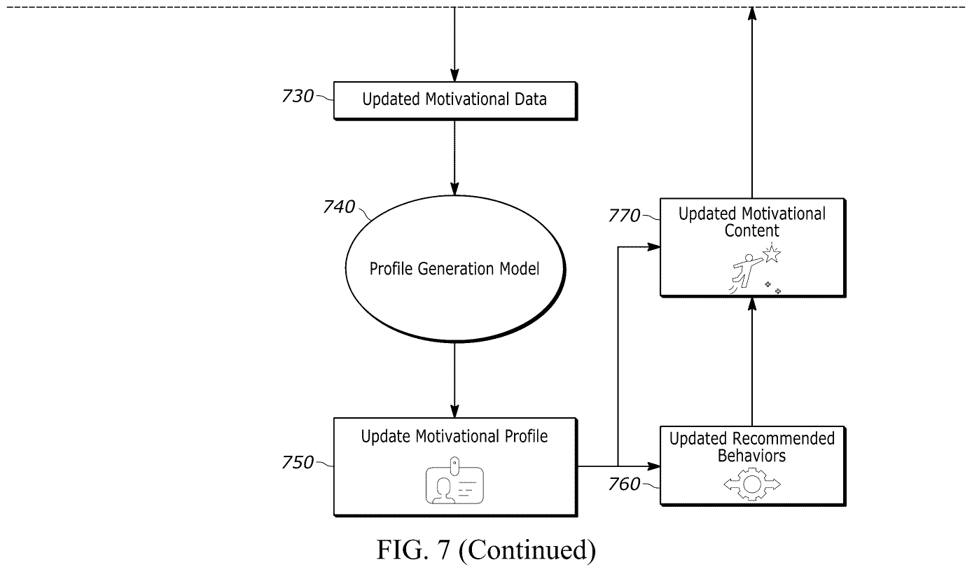

Additionally, the motivational content server may be configured to monitor the user for compliance with the recommended behavior to determine whether any changes to how the motivational content 230 is generated may lead to increased compliance. Accordingly, the motivational content server may obtain compliance data 232 indicative of whether the user performed the recommended behavior 222. The motivational content server may then update the motivation data 210 and/or the motivational profile 218 based on the compliance data 232 such that the behavior generation model 220 can generate or select more tailored recommended behaviors 222 and/or the motivation generation machine learning model 224 generates more tailored motivational content 230. More details about updating the motivational profile based on compliance data will be explained below with regards to FIG. 7.

FIG. 3 depicts a combined block and logic diagram 300 for obtaining motivational data 340 (such as the motivational data 210), according to one embodiment. The techniques described with respect to FIG. 3 may be implemented by a motivational content server (e.g., the motivational content server 110).

In some embodiments, the motivational content server may derive the motivational data 340 based on data obtained from a user device of the user. For example, the motivational content server may obtain user input data 306, such as survey/questionnaire data 324 (e.g., as obtained via a GUI presented by the motivation application 164) and/or location data 326 (e.g., data obtained from the user device operating system upon the user providing permission to the motivation application 164). As another example, the user input data 306 may include data 308 derived from other applications executing on the user device. For instance, the data 308 may include fitness data 326 generated by a fitness tracking application (for which the user provided permission to share with the motivational application 164).

As another example, the motivational content server may also obtain health records provided with the user's consent. For example, the user's health records 304 may include but are not limited to test results 320 or physiological data 322.

As yet another example, the motivational content server may also obtain content consumption and creation data 302. For example, the content consumption and creation data 302 may include but are not limited to content consumed or created on all kinds of social media platforms 310, through chats or messages 312 and emails 314, or in the forms of video or audio 316 and books or journals 318. To this end, the content consumption and creation data 302 may indicate topics and/or categories of interest to the user such that the motivational content server can generate motivational data the is more likely to resonate with the user. As one example, if the content consumption and creation data 302 indicates that the user enjoys cooking their own meals, the motivational content may relate to a behavior associated with healthy cooking. As another example, if the content consumption and creation data 302 indicated that the user has watched a particular TV show, the motivational content server may generate motivational content that references characters from that TV show.

As illustrated, after obtaining the data from the user device, the motivational content server may utilize a machine learning model 330 to process the various sources of data described above and generate motivational data 340. More particularly, the machine learning model 330 may be configured to analyze the obtained data to derive health goals 342 and preferences 346. For example, the machine learning model 330 may be configured to analyze the survey data 324 and/or the health records 304 to identify the health goals 342. As another example, the machine learning model 330 may be configured to analyze the content consumption and creation data 302 to derive the content preferences 350 and/or delivery method preferences 348 (e.g., at time periods associated with certain content consumption characteristics). While the term “machine learning model” is used, in other embodiments, the model 330 may instead be a rules-based model.

FIG. 4 depicts a combined block and logic diagram 400 for associating a user with a motivational profile 430 (e.g., the motivational profile 218) based on the motivational data 410 (e.g., the motivational data 340), according to one embodiment. The techniques described with respect to FIG. 4 may be implemented by a motivational content server (e.g., the motivational content server 110).

In one embodiment, the motivational content server may input the motivational data 410 into a profile generation model 420 where the profile generation model 420 assigns the user a motivational profile 430. In some embodiments, the profile generation model 420 may include a profile classification algorithm 424 to assign the user a predefined motivational profile 422. The profile classification algorithm 424 may include a neural network trained to map the motivational data to one or more predefined motivational profiles 422. For example, profile classification algorithm 424 may include a set of interconnected nodes, layers, trained parameter values (e.g., multiplicative weights, additive bias, etc.), etc. The trained parameters are set via backpropagation techniques in a training process that uses historical user profile mappings as ground truth in a training process.

As another example, the profile generation model 420 may include a custom profile generation algorithm 426 to create a new motivational profile and/or modify a predefined motivational profile 422. Like the profile classification algorithm 424, the custom profile generation algorithm 426 may include a neural network. However, the custom profile generation algorithm 426 may divide the motivational profile into subparts. In some embodiments, the motivational content server associates the subparts with respective sets of predefined options such that the custom profile generation algorithm 426 can generate a composite motivation profile 430 based on the custom profile generation algorithm 426 mapping the motivational data 410 to the options for each subpart. In these embodiments, the custom profile generation algorithm 426 may also be trained via backpropagation techniques based on historical mappings for each subpart. In other embodiments, the custom profile generation algorithm 426 may implement a generative AI algorithm to extract data from the motivational data 410 and generate customized data for each subpart. In these embodiments, the custom profile generation algorithm 426 may include a prompt template configured with a set of rules that define how the generative AI algorithm is to analyze the motivational data 410 to generate the customized data.

FIG. 5 depicts a combined block and logic diagram 500 for determining recommended behaviors 530 based on the motivational profile, according to one embodiment. The techniques described with respect to FIG. 5 may be implemented by a motivational content server (e.g., the motivational content server 110).

In one embodiment, the motivational content server may input the motivational profile 510 into a behavior generation model 520 to generate the recommended behaviors 530. As described above, the motivational content server may associate each health goal with a set of predefined behaviors 522 that one can perform in furtherance of the health goal. In some embodiments, the possible behaviors are generalized (e.g., a behavior to exercise, a recommended sleep or meal schedule, limit TV viewing, meditate, etc.).

In this embodiment, the behavior generation model 520 may implement a behavior selection algorithm 524 configured to select from the predefined recommended behaviors 522. The behavior selection algorithm 524 may include a set of interconnected nodes, layers, trained parameter values (e.g., multiplicative weights, additive bias, etc.), etc. The trained parameters are set via backpropagation techniques in a training process that uses historical data in a training process. In particular, the historical data may include motivational profile data, selected behavior recommendations, and compliance data associated with the recommendation. Accordingly, the training process may increase associations between motivational profile data and recommended behaviors that resulted in user compliance and decrease associations between motivational profile data and recommended behaviors that did not result in user compliance. As a result, the behavior selection algorithm 524 is trained to learn which behaviors are most likely to be performed for a given motivational profile 510.

In other embodiments, the possible behaviors are templates that can be modified based on the preference data of the motivational profile 510. In these embodiments, the template behavior may be customized using a custom behavior generation algorithm 526. For example, the custom behavior generation algorithm 526 may determine that a user enjoys passing through a particular park in their neighborhood and generate a customized behavior to go for a run or walk that passes through the park. As a further refinement, if the motivational profile indicates that user has a dog, the customized behavior may be framed as walking the dog, instead of performing exercise. As another example, the motivational profile 510 may include content preference data that indicates the user recently discussed Italy. In this example, the custom behavior generation algorithm 526 may generate a customized behavior to cook a particular healthy Italian dish (as opposed to a general recommendation to eat a healthy meal). Accordingly, the custom behavior generation algorithm 526 may be trained in a similar manner as the behavior selection algorithm 524, except with a more refined focus on aspects of the motivational profile 510 such that the custom behavior generation algorithm 526 is able to have knowledge of additional characteristics of users (e.g., content consumption preferences, exercise preferences, personality traits, etc.) that enable the behavior generation model 520 to generate customized recommended behaviors 530 that are even more likely to result in user compliance.

Regardless of the model type, the motivational content server may compare the recommended behaviors to the motivational profile 510 to ensure that the user is capable of performing the recommended behaviors. For example, if the user has a leg injury, the motivational content server would avoid providing recommendations to go on a run. Accordingly, if the user is unable to perform the recommended action, the motivational content server may select a new behavior and/or generate a new custom behavior.

FIG. 6 depicts a combined block and logic diagram for generating user-specific motivational content based on the recommended behaviors, including an example motivational content prompt, according to one embodiment. The techniques described with respect to FIG. 6 may be implemented by a motivational content server (e.g., the motivational content server 110).

In one embodiment, the motivational content server may input the motivational profile 612 and the recommended behaviors 614 into a motivation generative AI module 620 to generate motivational content 650. As described above, the motivation generative AI model 620 may maintain a motivational content prompt template (such as the template 128) that forms the basis of a prompt input into a generative AI model 640 (e.g., a pre-trained generative AI model, such as a GPT model and/or diffusion model) to generate the motivational content 650.

As illustrated, the motivation generation ML module 620 may populate the prompt template to generate the motivational content prompt 630. The motivational content prompt 630 may include several sets of rules and/or instructions for how to generate the motivational content 650.

The motivational content server may then input a motivational content prompt 630 into the generative AI model 640 where the generative AI model 640 is configured to analyze the motivational content prompt 630 to generate and provide the user specific motivational content 650. For example, the prompt 630 may include user data 632 derived from the motivational profile 612 (e.g., data about their health goals and their personal interests) and the recommended behaviors 614 (e.g., indication of the behavior the motivational content 650 is intended to motivate). Accordingly, the motivation generative AI model 620 may generate the user data 632 portion of the prompt 630 prior to each call to the generative AI model.

Additionally, the prompt 630 includes instructions for how to analyze the user data 632. In some embodiments, the analysis instructions 634 may be fixed across calls to the generative AI model 640. In other embodiments, the predefined motivational profiles may include respective sets of analysis instructions 634 that have specific instructions tailored to the personality type associated with the predefined motivational profiles. For example, the instructions 634 may include instructions for a tone or style to which the personality type is likely to respond.

As illustrated, the prompt 630 may also include instructions 636 for formatting the motivational content. For example, the instructions 636 may detail a preferred format for the motivational content (an image, a video, website content, audio recording, etc.) derived from the motivational profile 612. In some embodiments, the instructions 636 may identify a content database storing the content type and a query for obtaining a particular object from the content database. It should be appreciated that the instructions 632-636 are only example sets of instructions that may be included in the prompt 630 and in other embodiments, the prompt 630 may include additional set of instructions.

Regardless, after the motivation generative AI model 620 generates the prompt 630, the motivation generative AI model 620 may input the prompt 630 into the generative AI model 640 which generates the motivational content 650 in accordance with the instructions set forth in the prompt 630. The motivation generative AI model 620 may then utilize the output of the generative AI model 640 as the motivational content 650.

After obtaining the motivational content 650, the motivational content server may provide the motivational content 650 to the user device for presentation thereat.

FIG. 7 depicts a combined block and logic diagram 700 for updating the motivational content based on compliance data, according to one embodiment. More particularly, the diagram 700 may relate to tracking the user after the provision of motivational content 714 (e.g., the motivational content 650) intended to motivate the user to perform a recommended behavior 712 (e.g., the recommended behaviors 614). The techniques described with respect to FIG. 6 may be implemented by a motivational content server (e.g., the motivational content server 110).

As illustrated, in one embodiment, the motivational content server may obtain compliance data 720 indicative of whether the user performed the recommended behavior 714. The compliance data 720 may vary depending on the particular behavior and various permissions the user has provided the motivational content server related to tracking. For example, for some behaviors (e.g., cooking a meal, going to sleep by a certain time, etc.) the compliance data may be in the form of a survey or questionnaire data 722 that the user self-provides via a GUI of the user device. For other behaviors, compliance data 724 may be provided via one or more applications executing on the user device. For example, a fitness tracking application may determine that the user went on a run following a particular route, or a user device operating system may indicate that the user limited phone usage to a predetermined amount. Still other types of compliance data 726 may indicate whether a user consumed recommended content to help the user learn more about their health goal. For example, the user device may track whether the user clicked on a link and/or viewed content hosted at the link. As yet another type of compliance data 728 may be derived from health records (e.g., an indication of whether or not the recommended actions are providing measurable progress toward the health goal, an indication confirming the user visited a doctor, etc.).

After obtaining the compliance data 720, the motivational content server may determine whether the compliance data 720 indicates that the user performed the recommended actions 714. The motivational content server may then generate updated motivational data 730. More particularly, the content motivational server may update the user's motivational data (e.g., the motivational data 210) to include the compliance data 720 such that the motivational content server learns updated user motivations over time.

Based on the updated motivational data, the motivational content server may then input the updated motivational data into a profile generation model 740 (such as the profile generation model 420) to generate an updated motivational profile 750 for the user. For example, the profile generation model 740 may determine that particular types of motivational content have not led to user compliance and update the motivational profile to include an indication to avoid that type of motivational content.

As described above, the motivational content server may be configured to regularly provide motivational content to the user as they progress toward achieving their health goals. For example, in some embodiments, the motivational content server may be configured to generate new motivational content at a particular time each day. It should be appreciated that the motivational content server may wait until the next periodic interval before analyzing the compliance data 720 to increase the amount of time to comply with the recommended behavior. That said, for some behaviors that are to occur at a certain time, the motivational content server may determine compliance at the expiration of the corresponding time window.

In any event, after detecting the subsequent stimulus to generate motivational content, the motivational content server may then generate updated recommended behaviors (e.g., in the manner described by the diagram 500) and updated motivational content 770 (e.g., in the manner described by the diagram 600) based on the updated motivational profile 750. This process may repeat until the user has achieved their health goals.

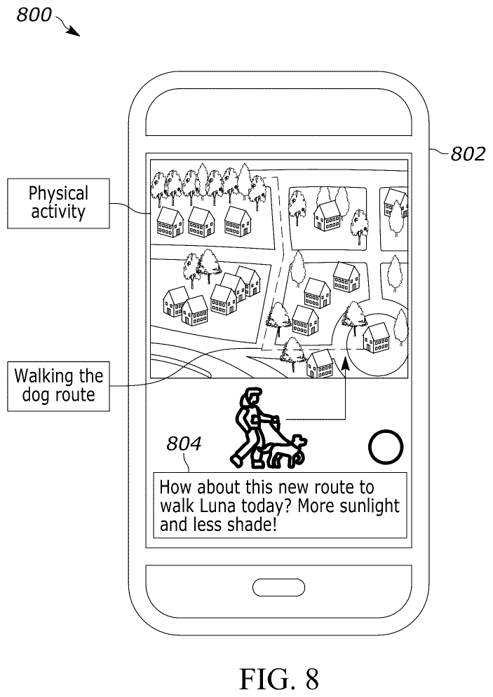

FIG. 8 depicts an example user device display of the motivational content, according to one embodiment. In one embodiment, a user device 802 may be configured to display the motivational content with an illustration of a map showing a route to walk the user's dog Luna and a message in encouraging tone to suggest a recommended physical activity of walking the dog along the recommended route. Here, the preferred delivery format is image and message. The message reflects the user's preference data included in their motivational profile (e.g., walking the dog with sunlight instead of under shades).

### Example Methods

FIG. 9 depicts flow diagram of an example computer-implemented method 900 as may be implemented by system 100 of FIG. 1, for generating personalized motivational content based on user-specific data and goals, according to one embodiment. One or more steps of method 200 may be implemented as a set of instructions stored on a computer-readable memory (e.g., memorie(s) 120) and executable on one or more processors (e.g., processor 112).

The method 900 may begin at block 902 when the motivational content server (e.g., 110) obtains motivational data (e.g., 210, 340, 410, 730), indicating a health goal (e.g., 212, 342) for a user. For instance, this may include performing one or more of detecting user data input (e.g., 206, 306), obtaining user health data from health records (e.g., 204, 304), and extracting user data based on content consumption and creation data associated with the user (e.g., 202), as illustrated by FIG. 2.

The method 900 may further include, at block 904, associating the user with a motivational profile (e.g., 218, 430, 510, 750) based on the motivational data. For instance, this may include inputting the motivational data into a profile generation model (e.g., 122, 216, 420, 740), matching the motivational data with a particular profile of the one or more predefined motivational profiles (e.g., 422), and assigning the particular profile to the user, as illustrated in FIG. 4. In some instances, this may include inputting the motivational data into a profile generation model configured to analyze the motivational data to generate a customized motivational profile to the user based on the motivational data.

At block 906, the method 900 may further include determining one or more recommended behaviors (to achieve the indicated health goal based on the motivational data. To determine the recommended behaviors (e.g., 222, 530, 614, 712, 760), the motivational content server may input the motivational profile into a behavior generation machine learning model (e.g., 124, 220, 520), where the behavior generation machine learning model is configured to analyze the input motivational profile to output the one or more recommended behaviors. In some instances, the motivational content server may associate the indicated health goal with a set of predefined recommended behaviors (e.g., 522), and select the one or more recommended behaviors from the set of predefined recommended behaviors, as illustrated by FIG. 5.

The method 900 may further include, at block 908, inputting the motivational profile and the one or more recommended behaviors into a motivation generation machine learning model (e.g., 224, 620) to obtain motivational content (e.g., 230, 650, 714, 770). For instance, as illustrated by FIG. 6, the motivational content server may input the motivational profile and the recommended behaviors into a generative AI model (e.g., 126, 228, 640), input a motivational content prompt (e.g., 226, 630) into the generative AI model. The generative AI model is configured to analyze the motivational content prompt to generate and provide the user-specific motivational content. The motivational content server may further determine a preferred format of the motivational content, and the format may include image, video, website, or recording. Additionally, the method 900 may include, at block 910, presenting the motivational content to the user.

The method 900 may further include obtaining updated motivational data indictive of compliance data (e.g., 232, 720) with regards to the recommended behavior and the motivational content and updating the motivational profile based on the compliance data. Additionally, the motivational content server may further detect a content generation stimulus (e.g., 219) to perform at least one of the following processes at a predetermined interval: (i) determining one or more recommended behaviors to achieve the indicated health goal based on the updated motivational profile, and (ii) inputting the updated motivational profile and the one or more recommended behaviors into the motivation generation machine learning model, as illustrated by FIG. 7.

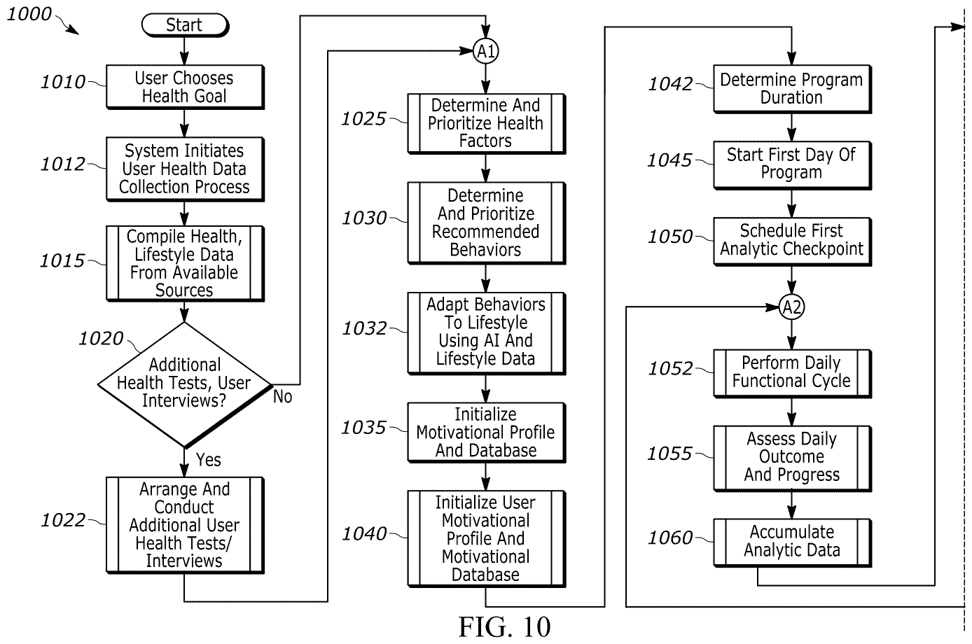

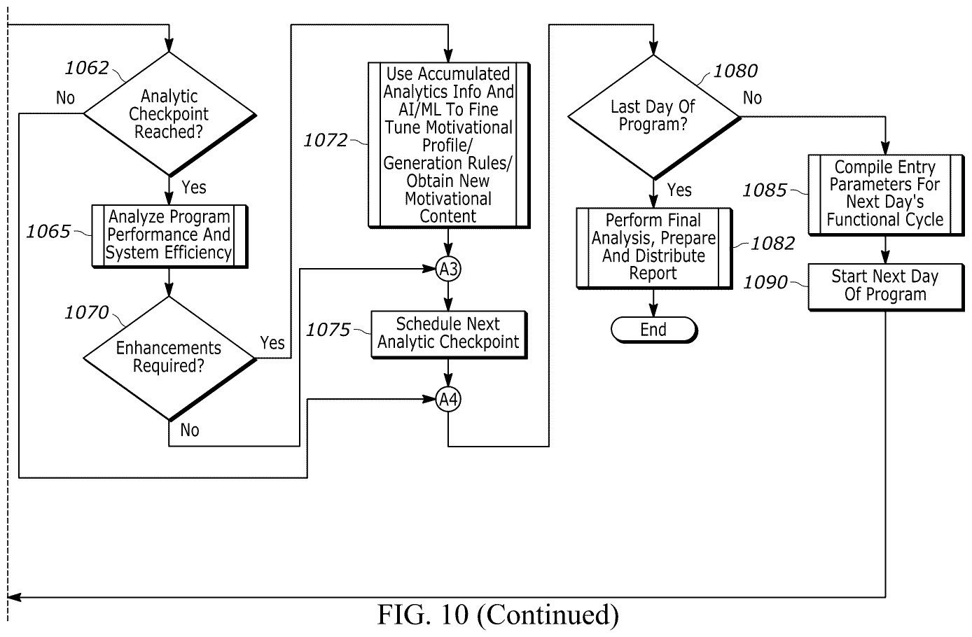

FIG. 10 depicts an example flow diagram for how the techniques described herein may be implemented, according to one embodiment. To start, the user chooses a health goal (1010), which initiates a user health data collection process (1012) which compiles health and lifestyle data from available sources (1015). This data may include the compliance data 720. There's a decision point (1020) for collecting additional health tests or user interviews; if “Yes”, these tests and interviews are arranged (1022), if “No”, then the process proceeds to determine and prioritize health factors (1025). Next, the process determines and prioritizes recommended behaviors (1030) (e.g., by applying the techniques of the diagram 500), then adapts these behaviors to the user's lifestyle using AI and lifestyle data (1032) to initialize a motivational profile and database for the user (1035-1040). In the next step, the program's duration is determined (1042), and then the first day of the program begins (1045). A first analytic checkpoint (e.g., a stimulus to generate motivational content) is then scheduled (1050).

Moving onto the next step, a recurring process starts with performing daily functional cycle (1052), assessing daily outcomes and progress (1055), and accumulating analytic data (1060). Next, an analytic checkpoint is reached (1062), prompting an analysis of both the program's performance and its efficiency (1065). If the system determines that enhancements are required (1070), the system may use accumulated analytics information and AI/ML to fine-tune or otherwise update the motivational profile, generation rules, or obtain new motivational content (1072). After the fine-tuning if completed or if no enhancements are required, the next step is to schedule the next analytics check point (1075). If the program reaches the last day, it performs a final analysis and prepares and distributes the report before ending the program. If it is not the last day of the program, the system continues to compile entry parameters for the next day's functional cycle (1085) and start the next day of the program (1090) with performing daily functional cycle (1052).

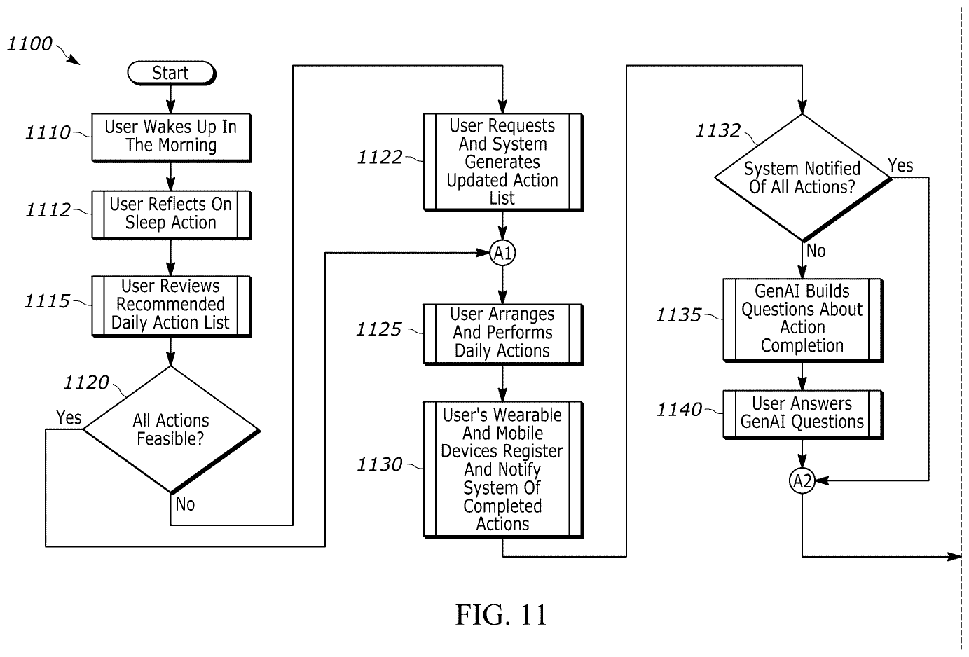

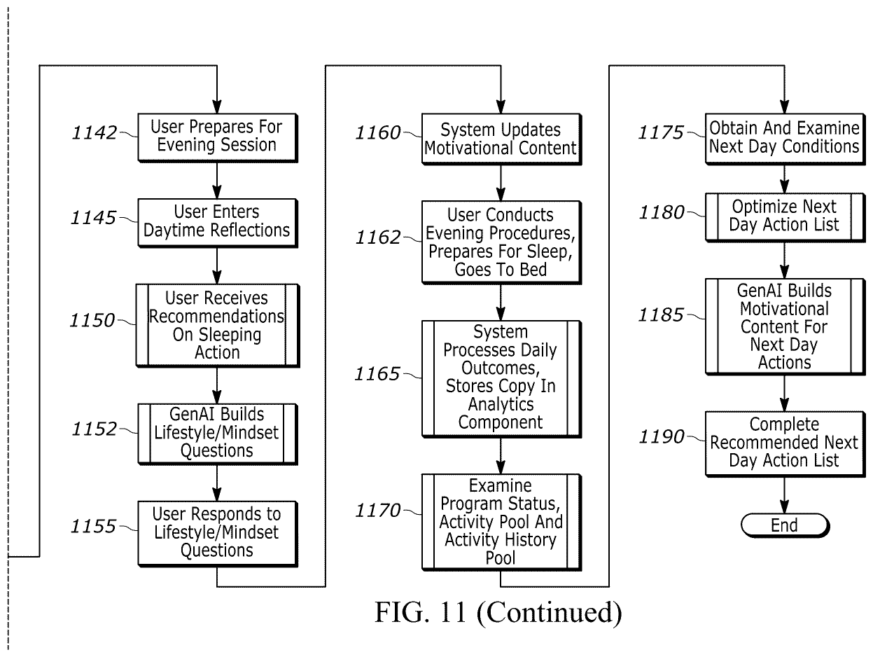

FIG. 11 depicts an example flow diagram for how the techniques described herein may be implemented on a daily schedule, according to one embodiment. The process starts with the user waking up in the morning (1110) and reflecting on the user's sleep action (1112) before reviewing a recommended daily action list (1115), such as a list included in the motivational content. The user checks if all actions on the list are feasible (1120), and if not, the user requests the system to generate an updated action list (1122). The user then arranges and performs daily actions (1125). During the day, the user's wearable and mobile devices register and notify the system of completed actions (1130). This process contains a decision point to check if the system is notified of all actions (1132). If so, it transitions to the next part of the process. If not, the system builds questions about action completion with GenAI (1135), and the user answers these questions (1140). Afterwards, the user prepares for an evening session (1142) and then enters daytime reflections (1145). In the next step, the user receives recommendations on sleeping action (1150). The system generates lifestyle/mindset questions with GenAI (1152) that the user responds to (1155), which prompts the system to update motivational content (1160). The user then conducts evening procedures, prepares for sleep, and goes to bed (1162).

In the next step, the system processes daily outcomes and stores a copy in an analytics component (1165) (e.g., by updating the user's motivational profile). The system then examines the program status, activity pool and activity history (1170) to obtain and examine conditions for the next day (1175), optimize the next day's action list (1080), builds motivational content for the next day's actions with GenAI (1085), and finally completes the recommended action list for the next day (1090) before ending the process.

### Additional Considerations

The following additional considerations apply to the foregoing discussion and the appended claims. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall with-in the scope of the subject matter of the present disclosure.

Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first set of one or more processors (e.g., in a first computing device) generates X and a distinct, second set of one or more processors (e.g., in a different, second computing device) independently generates Y; (2) implementations in which all processors in the set of one or more processors (e.g., all in the same device, or distributed among multiple devices) contribute to the generation of both X and Y; and (3) other variations.

Unless specifically stated otherwise, discussions in the present disclosure using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used in the present disclosure any reference to “one implementation” or “an implementation” means that a particular element, feature, structure, or characteristic described in connection with the implementation is included in at least one implementation or implementation. The appearances of the phrase “in one implementation” in various places in the specification are not necessarily all referring to the same implementation.

As used in the present disclosure, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs through the principles described herein. Thus, while particular implementations and applications have been illustrated and described, it is to be understood that the disclosed implementations are not limited to the precise construction and components disclosed in the present disclosure. Various modifications, changes and variations, which will be apparent to those skilled in the art, may be made in the arrangement, operation and details of the method and apparatus disclosed in the present disclosure without departing from the spirit and scope defined in the appended claims.

1. A computer-implemented method for providing recommended actions to motivate a user, the method comprising:
obtaining, by one or more processors, motivational data indicating a health goal for a user;
based on the motivational data:
associating, by the one or more processors, the user with a motivational profile; and
determining, by the one or more processors, one or more recommended behaviors to achieve the indicated health goal;
inputting, by the one or more processors, the motivational profile and the one or more recommended behaviors into a motivation generation machine learning model to obtain motivational content; and
presenting, via the one or more processors, the motivational content to the user.

2. The method of claim 1, wherein obtaining motivational data comprises:
performing, by the one or more processors, one or more of:
detecting user data input;
obtaining user health data from health records; and
extracting user data based on content consumption and creation data associated with the user.

3. The method of claim 2, wherein user data input comprises:
user input data obtained from the user; and
data automatically captured by one or more applications executing on a personal electronic device associated with the user, wherein the one or more applications include one or more of a fitness application and a location tracking application.

4. The method of claim 3, wherein associating the user with a motivational profile comprises:
inputting, by the one or more processors, the motivational data into a profile generation model, wherein the profile generation model is associated with one or more predefined motivational profiles;
matching, by the profile generation model, the motivational data with a particular profile of the one or more predefined motivational profiles; and
assigning, by the profile generation model, the particular profile to the user.

5. The method of claim 3, wherein associating the user with a motivational profile comprises:
inputting, by the one or more processors, the motivational data into a profile generation model configured to analyze the motivational data to generate a customized motivational profile to the user based on the motivational data.

6. The method of claim 1, wherein determining the one or more recommended behaviors comprises:
inputting, by the one or more processors, the motivational profile into a behavior generation machine learning model, wherein the behavior generation machine learning model is configured to analyze the input motivational profile to output the one or more recommended behaviors.

7. The method of claim 6, wherein to output the one or more recommended behaviors, the behavior generation machine learning model is configured to:
associate the indicated health goal with a set of predefined recommended behaviors; and
select the one or more recommended behaviors from the set of predefined recommended behaviors.

8. The method of claim 1, wherein obtaining the motivational content to the user comprises:
inputting, by the one or more processors, a motivational content prompt generated based on the motivational profile and the recommended behaviors into a generative AI model included in the motivation generation machine learning model, wherein the generative AI model is configured to analyze the motivational content prompt to generate user-specific motivational content; and
providing, by the one or more processors, the motivational content.

9. The method of claim 8, wherein generating the motivation content further comprises:
determining, by the one or more processors, a preferred format of the motivation content, wherein the format includes at least one selection from the following: an image, a video, website content, or an audio recording.

10. The method of claim 1, further comprising:
obtaining, by the one or more processors, updated motivational data indicative of compliance data with regards to the recommended behavior and the motivational content; and
updating, by the one or more processors, the motivational profile based upon the compliance data.

11. The method of claim 10, wherein at least one of (i) determining one or more recommended behaviors to achieve the indicated health goal based on the updated motivational profile, and (ii) inputting the updated motivational profile and the one or more recommended behaviors into the motivation generation machine learning model is performed at a predetermined interval.

12. A computing system for optimizing customer service efficiency, the system comprising:
one or more processors; and
one or more memories storing processor-executable instructions that, when executed by the one or more processors, cause the one or more processors to:
obtain motivational data indicating a health goal for a user;
based on the motivational data:
associate the user with a motivational profile; and
determine one or more recommended behaviors to achieve the indicated health goal;
input the motivational profile and the one or more recommended behaviors into a motivation generation machine learning model to obtain motivational content; and
present the motivational content to the user.

13. The computing system of claim 12, wherein to obtain the motivational data, the processor-executable instructions cause the one or more processors to:
perform one or more of:
detecting user data input;
obtaining user health data from health records; and
extracting user data based on content consumption and creation data associated with the user.

14. The computing system of claim 13, wherein to obtain the motivational data, the processor-executable instructions cause the one or more processors to:
input the motivational data into a profile generation model, wherein the profile generation model is associated with one or more predefined motivational profiles;
match, by the profile generation model, the motivational data with a particular profile of the one or more predefined motivational profiles; and
assign, by the profile generation model, the particular profile to the user.

15. The computing system of claim 12, wherein to obtain the motivational data, the processor-executable instructions cause the one or more processors to:
input the motivational profile into a behavior generation machine learning model, wherein the behavior generation machine learning model is configured to analyze the input motivational profile to output the one or more recommended behaviors.

16. The computing system of claim 15, wherein the processor-executable instructions cause the one or more processors to output the one or more recommended behaviors, the behavior generation machine learning model is configured to:
associate the indicated health goal with a set of predefined recommended behaviors; and
select the one or more recommended behaviors from the set of predefined recommended behaviors.

17. The computing system of claim 12, wherein the processor-executable instructions cause the one or more processors to obtain the motivational content to the user comprises:
inputting, by the one or more processors, a motivational content prompt generated based on the motivational profile and the recommended behaviors into a generative AI model included in the motivation generation machine learning model, wherein the generative AI model is configured to analyze the motivational content prompt to generate user-specific motivational content; and
providing, by the one or more processors, the motivational content.

18. The computing system of claim 12, wherein the processor-executable instructions cause the one or more processors to further:
obtain updated motivational data indicative of compliance data with regards to the recommended behavior and the motivational content; and
update the motivational profile based upon the compliance data.

19. The computing system of claim 18, wherein the processor-executable instructions cause the one or more processors to perform at a predetermined interval at least one of (i) determining one or more recommended behaviors to achieve the indicated health goal based on the updated motivational profile, and (ii) inputting the updated motivational profile and the one or more recommended behaviors into the motivation generation machine learning model.

20. One or more non-transitory computer-readable media storing processor-executable instructions that, when executed by one or more processors, cause the one or more processors to:
obtain motivational data indicating a health goal for a user;
based on the motivational data:
associate the user with a motivational profile; and
determine one or more recommended behaviors to achieve the indicated health goal;
input the motivational profile and the one or more recommended behaviors into a motivation generation machine learning model to obtain motivational content; and
present the motivational content to the user.
